(19) United States
(12) Patent Application Publication
(10) Pub. No.: US 2007/0191627 A1
(43) Pub. Date: Aug. 16, 2007

(54) Stilbene derivatives and their use in medicaments

(76) Inventors: Thomas Szekeres, Wien (AT); Norbert Handler, Wien (AT); Walter Jager, Pressbaum (AT); Arkadiusz Marek Murias, Wien (AT); Thomas Erker, Wien (AT)

Correspondence Address: YOUNG & THOMPSON, 745 SOUTH 23RD STREET, 2ND FLOOR, ARLINGTON, VA, 22202, US

(21) Appl. No.: 10/567,774

(22) PCT Filed: Aug. 9, 2004
(86) PCT No.: PCT/AT04/00279

§ 371 Date: Jan. 12, 2007

(30) Foreign Application Priority Data
Aug. 14, 2003 (AT) A 1285/2003

(51) Int. Cl. C07C 69/00; C07C 43/20

(52) U.S. Cl. 560/138; 568/660

(57) ABSTRACT

The invention relates to stilbene derivatives of general formula (I), in which at least four of the substituents R₁ to R₆ do not represent hydrogen. The substituents are effective radical captors, anti-tumour active ingredients and selective cyclooxygenase-2 inhibitors.

The invention relates to stilbene derivatives of general formula I 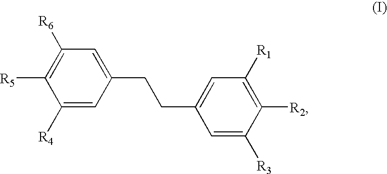
in which R₁ to R6, which are the same or different, mean hydrogen, OH—, —OD or —OR₇, in which R₇ is a C₁ to C₃-alkyl group or a C₂ to C₄-carboxyl group, provided that at least four (4) of the substituents R₁ to R₆ have a meaning different from hydrogen.

In addition, the invention relates to pharmaceutical agents that contain at least one of the compounds of general formula I.

Moreover, the invention extends to the use of the compounds of general formula I for the production of pharmaceutical agents.

The compounds according to the invention are pharmaceutically valuable active ingredients since they act as free-radical scavengers, as anti-tumor substances, and/or as selective cyclooxygenase-2 inhibitors (COX 2).

In the material on hand, 3,3′,4,4′,5,5′-hexahydroxystilbenes and their ethers and esters are considered; and in particular the methyl, ethyl and propyl ether are considered as ethers, and in particular those of formic acid and acetic acid are considered as esters.

In addition, deuterated 3,3′,4,4′,5,5′-hexahydroxystilbenes—in whose —OH groups deuterium, instead of hydrogen, is contained, and thus OD groups are present instead of OH groups—are considered within the scope of the invention.

3,3′,4,4′,5,5′-Hexahydroxystilbene and 3,3′,4,4′,5,5′-hexamethoxystilbene are especially considered within the scope of the invention. These compounds have proven their value as active ingredients with advantageous properties as free-radical scavengers and anti-tumor substances as well as as highly-selective cyclooxygenase-2 inhibitors.

More generally speaking, in the material on hand, the following compounds are considered:

E and Z forms of: 3,3′,4,4′,5,5′-hexahydroxystilbene, but also 3,3′,4,5,5′-pentahydroxystilbene, 3,3′,4,4′,5-pentahydroxystilbene, 3,4,4′,5,5′-pentahydroxystilbene, 3,4,4′,5-tetrahydroxystilbene, 3,3′,5,5′-tetrahydroxystilbene, 3,3′,4′,5′-tetrahydroxystilbene and ethers (methoxy, ethoxy, propoxy) and esters (formate, acetate) of these compounds; these are, for example, tetra-, hexa- and pentamethoxy-stilbene derivatives of the above-mentioned structures, mixed hydroxy- and methoxy-stilbene derivatives, but also deuterated (D instead of H) analogs of the above-mentioned structures as highly selective COX 2 inhibitors with significantly lower inhibition action on COX 1 than on COX 2.

Pharmaceutical agents that contain the above-mentioned stilbene derivatives of formula I are suitable for the prevention and the treatment of various diseases, including tumor diseases, and they are dependent on the properties of active ingredients as free-radical scavengers and for all syndromes and diseases that can be treated by the use of cyclooxygenase-2 inhibitors.

### BACKGROUND OF THE INVENTION

Many naturally-occurring substances, such as flavonoids or phenols, can prevent the development of a number of diseases or are used effectively in the treatment of various diseases, such as, for example, tumor diseases or cardiovascular diseases. These substances can be found in various plant extracts, spice mixtures or plants, such as berries, grapes, peanuts, or else in wine and were already used, for example, in Indian or Chinese medicine.

Resveratrol (3,5,4′-trihydroxystilbene) is the most carefully examined polyphenolic substance. It is formed by grapes and can be found in wine. Resveratrol effectively inhibits the growth of tumor cells and is regarded as responsible for the so-called “French paradox” (the fact that the probability of developing coronary heart disease is reduced by 40% in France compared to all other European countries). A number of cellular actions, such as the inhibition of the cyclooxygenase (COX) activity (Subbaramaiah, K.; Chung, W. J.; Michaluart, P.; Telang, N.; Tanabe, T.; Inoue, H.; Jang, M.; Pezzuto, J. M.; Dannenberg, A. J. Resveratrol Inhibits Cyclooxygenase-2 Transcription and Activity in Phorbol Ester-Treated Human Mammary Epithelial Cells. J Biol Chem. 1998 Aug. 21; 273 (34):21875-82), inhibition of the ribonucleotide reductase activity (Fontecave, M.; Lepoivre, M.; Elleingand, E.; Gerez, C.; Guittet, 0. Resveratrol, A Remarkable Inhibitor of Ribonucleotide Reductase. FEBS Lett. 1998 Jan. 16; 421 (3):277-9) or induction of NFkappaB (Tsai, S. H.; Lin-Shiau, S. Y.; Lin, J. K. Suppression of Nitric Oxide Synthase and the Down-Regulation of the Activation of NFkappaB in Macrophages by Resveratrol. Br J Pharmacol. 1999 February; 126 (3):673-80) were described for resveratrol. A selective inhibition of the cyclooxygenase-2 (COX 2) could not be shown for resveratrol. This is also not the case: resveratrol inhibits both isoenzymes (COX 1 and COX 2) with comparable effectiveness.

Various analogs of resveratrol are mentioned in WO 01/21165 A1 as anti-tumor substances. The substance, the synthesis and the use of 3,5,4,4′,5,5′-hexahydroxy, hexamethoxy and related analogs of resveratrol are not disclosed in WO 01/21165 A1. The hexahydroxy compound, however, was unexpectedly a very effective inhibitor of the growth of human tumor cells. In addition, this substance, but also the hexamethoxy compound, has shown a selective inhibition for COX 2.

For other resveratrol analogs, Ghai et al. (Ghai et al. WO 01/21165 A1, Lu, J.; Ho, C. H.; Ghai, G.; Chen, K. Y. Resveratrol Analog, 3,4,4′,5-Tetrahydroxystilbene, Differentially Induces Pro-Apoptotic p53/Bax Gene Expression and Inhibits the Growth of Transformed Cells But Not their Normal Counterparts. Carcinogenesis. 2001 February; 22 (2):321-8) describe anti-tumor activity, but the selective inhibition of COX 2 is not mentioned.

Two isoenzymes of the cyclooxygenase were identified in recent years. COX 2 is the inducible form, which must also be inhibited to inhibit inflammations, pains, etc. The inhibition of COX 1 is partially regarded as responsible for the side effects of so-called non-steroidal anti-inflammatory medications (NSAIDs), such as, for example, aspirin. Therefore, selective inhibitors of COX 2 were developed. As a result, the side effects of NSAIDs (mainly gastrointestinal problems) can be minimized and the effectiveness of the medications can be improved. Until now, some few highly selective inhibitors of COX 2 were described. Some of the medications are approved. The indications for the use of COX 2 inhibitors are further listed below. They now comprise a number of syndromes and diseases.

Highly selective COX 2 inhibitors can be used, for example, for the treatment of the following syndromes and diseases:

Anti-tumor action

Treatment and prevention of malignant growths

Induction of apoptosis (programmed cell death)

Inhibition of NFkappaB

Use in the combination with radiation therapy

Use in the combination with other substances in chemotherapy

Reduction of the invasiveness and the metastatic potential of tumors

Fever-reducing (anti-pyretic) action

Inhibition of uterine contractions

Anti-inflammatory action

Treatment of asthma

Treatment of osteoarthritis and rheumatoid arthritis

Enhancing action on bone repair

Prevention and treatment of connective tissue diseases and bone diseases including osteoporosis

Antiestrogenic effects (treatment for tumor prevention and treatment of menopausal and post-menopausal symptoms)

Treatment of glaucoma

Pain reduction

Reduction of edemas

Anti-angiogenetic action, i.e., action on angiogenesis (inhibition of the formation of blood vessels)

Inhibition of platelet aggregation

Action on NO synthase

Prevention of cardiovascular diseases (vascular diseases)

Prevention and treatment of myocardial infarction

Prevention and treatment of diabetes and diabetes complications

Prevention of ischemically proliferative retinopathy

Prevention of reperfusion damage

Antiviral activity

Antibacterial activity

Antimycotic activity

Treatment against other pathogens such as: malaria treatment

Treatment of sickle cell anemia

Treatment of various skin diseases (also local topical use), such as, e.g., psoriasis

Treatment of actinic keratosis

Prevention and treatment of Helicobacter pylori gastritis

Prevention and treatment of Parkinson's disease

Treatment of amyotrophic lateral sclerosis

Treatment of multiple sclerosis

Treatment of Alzheimer's disease

Below, examples of the production of compounds of general formula I are presented, whereby the Horner-Emmons-Wadsworth reaction was used for the synthesis of methoxystilbenes.

### EXAMPLE 1

3,3′,5-Trimethoxystilbene. In a dry reaction flask, 10 mmol (2.58 g) of diethyl-(4-methoxybenzyl)phosphonate was cooled under argon to 0° C. Then, 10 ml of dry DMF, 20 mmol (1.12 g) of sodium methoxide and 10 mmol (1.661 g) of 3,5-dimethoxybenzaldehyde were added. The mixture was stirred at room temperature for one hour and then heated under argon for 1.5 hours to 100° C. Then, the solution was transferred into a beaker with 250 ml of ice water. The precipitate was filtered off and recrystallized from ethanol (70%).

Yield: 1.59 g (59%).

### EXAMPLE 2

3,4,4′,5-Tetramethoxystilbene was synthesized from: 10 mmol (2.58 g) of diethyl-(4-methoxybenzyl)-phosphonate, 20 mmol (1.12 g) of sodium methoxide and 10 mmol (1.962 g) of 3,4,5-trimethoxybenzaldehdye in the above-described way.

Yield: 1.65 g (55%);

Flash point=157° C.

¹H-NMR (200 MHz, CDCl₃): δ 7.44 (d, J=8.8 Hz, 2H), 6.98 (d, J=16.3 Hz, 1H), 6.91-6.83 (m, 3H), 6.71 (s, 2H), 3.90 (s, 6H), 3.86 (s, 3H), 3.82 (s, 3H). ¹³C-NMR (50 MHz, CDCl₃): δ 159.2, 153.3, 137.7, 133.3, 129.9, 127.6, 127.5, 126.4, 114.1, 103.2, 60.9, 56.0, 55.2.

MS m/z 300 (M⁺, 100%).

Anal (C₁₈H₂₀O₄) C, H.

### EXAMPLE 3

3,3′,5,5′-Tetramethoxystilbene was synthesized as described in Example 1 from: 10 mmol (2.88 g) of diethyl-(3,5-dimethoxybenzyl)phosphonate, 20 mmol (1.12 g) of sodium methoxide and 10 mmol (1.661 g) of 3,5-dimethoxybenzaldehyde.

Yield: 1.56 g (52%).

### EXAMPLE 4

3,3′,4′,5-Tetramethoxystilbene was synthesized as described in Example 1 from: 10 mmol (2.48 g) of diethyl-(3,5-dimethoxybenzyl)phosphonate, 20 mmol (1.12 g) of sodium methoxide and 10 mmol (1.661 g) of 3,4-dimethoxybenzaldehyde.

Yield: 1.65 g (55%).

### EXAMPLE 5

3,3′,4,5,5′-Pentamethoxystilbene was synthesized as described in Example 1 from: 10 mmol (2.48 g) of diethyl-(3,5-dimethoxybenzyl)phosphonate, 20 mmol (1.12 g) of sodium methoxide and 10 mmol (1.962 g) of 3,4,5-trimethoxybenzaldehyde.

Yield: 1.94 g (59%).

### EXAMPLE 6

3,3′,4,4′,5,5′-Hexamethoxystilbene was synthesized as described in Example 1 from: 10 mmol (3.18 g) of diethyl-(3,4,5-trimethoxybenzyl)phosphonate, 20 mmol (1.12 g) of sodium methoxide and 10 mmol (1.962 g) of 3,4,5-trimethoxybenzaldehyde.

Yield: 1.76 g (49%);

Flash point=215° C.

¹H-NMR (200 MHz, CDCl₃): δ 6.94 (s, 2H), 6.74 (s, 4H), 3.92 (s, 12H), 3.87 (s, 6H). ³C-NMR (50 MHz, CDCl₃): δ 153.3, 137.8, 132.8, 128.0, 103.3, 60.9, 56.0, MS m/z 360 (M⁺, 100%). Anal. (C₂₀H₂₄O₆) C, H.

### EXAMPLE 7

3,4,4′,5-Tetrahydroxystilbene: In a dry reaction flask, 2.5 mmol (0.750 g) of 3,4,4′,5-tetramethoxystilbene was dissolved under argon in methylene chloride and cooled to −30° C. Then, 15 mmol (15 ml of 1 M solution in methylene chloride) of boron tribromide solution was added drop by drop. The solution was heated to room temperature and stirred for 24 hours. The reaction was stopped by slow addition of saturated NaHCO₃ solution. Then, the solution was stirred for another 30 minutes, and methylene chloride was evaporated; the aqueous phase was acidified with 2N HCl. After EtOAc was added, the mixture was extracted. The organic phase was dried on Na₂SO₄, and the solvent was removed by vacuum. The crystals were recrystallized from EtOH/water or pure water.

Yield: 0.335 g (55%);

Flash point=240° C. ¹H-NMR (200 MHz, d₆-DMSO): δ 8.78 (s, 4H), 7.35 (d, J=8.6 Hz, 2H), 6.76-6.72 (m, 4H), 6.47 (s, 2H). ¹³C-NMR (50 MHz, d₆-DMSO): δ 156.7, 146.1, 132.9, 128.5, 128.2, 127.4, 125.9, 125.1, 115.5, 105.2.

MS m/z 244 (M⁺, 100%).

Anal. (C₁₄H₁₂O₄) C, H.

### EXAMPLE 8

3,3′,5,5′-Tetrahydroxystilbene was synthesized from 2.5 mmol (0.750 g) of 3,3′,5,5′-tetramethoxystilbene as described in Example 7.

Yield: 0.366 g (60%);

Flash point>320° C.

¹H-NMR (200 MHz, d₆-DMSO): δ 9.24 (s, 4H), 6.84 (s, 2H), 6.41 (d, J=2.0 Hz, 4H), 6.16 (t, J=1.9 Hz, 2H). ¹³C-NMR (50 MHz, d₆-DMSO): δ 158.5, 138.7, 128.4, 128.4, 104.6, 102.2.

MS m/z 244 (M⁺, 100%).

Anal. (C₁₄H₁₂O₄) C, H.

### EXAMPLE 9

3,3′,4′,5-Tetrahydroxystilbene was synthesized from 2.5 mmol (0.750 g) of 3,3′,4′,5-tetramethoxystilbene as described in Example 7.

Yield: 0.311 g(51%);

Flash point=236° C.

¹H-NMR (200 MHz, d₆-DMSO): δ 8.70 (s, 4H), 6.97 (s, 1H), 6.87-6.63 (m, 4H), 6.40 (d, 1.9 Hz, 2H), 6.20-6.19 (m, 1H). ¹³C-NMR (50 MHz, d₆-DMSO): δ 158.0, 144.9, 144.8, 138.9, 128.6, 127.8, 125.4, 118.3, 115.3, 112.7, 104.1, 101.6.

MS m/z 244 (M⁺, 100%).

Anal. (C₁₄H₁₂O₄) C, H.

### EXAMPLE 10

3,3′,4,5,5′-Pentahydroxystilbene was synthesized from 2.5 mmol (0.825 g) of 3,3′,4,5,5′-pentamethoxystilbene as described in Example 7.

Yield: 0.370 g (57%);

Flash point=252° C.

¹H-NMR (200 MHz, d₆-acetone): δ 8.00 (s, 5H), 6.89 (d, J=16.3 Hz, 1H), 6.76 (d, J=16.3 Hz, 1H), 6.64 (s, 2H), 6.51 (d, J=2.1 Hz, 2H), 6.28-6.25 (m, 1H). ¹³C-NMR (50 MHz, d₆-acetone): δ 160.2, 147.4, 141.4, 134.7, 130.5, 130.3, 127.7, 107.3, 106.3, 103.3.

MS m/z 260 (M⁺, 100%).

Anal (C₁₄H₁₂O₅).

### EXAMPLE 11

3,3′,4,4′,5,5′-Hexahydroxystilbene was synthesized from 2.5 mmol (0.900 g) of 3,3′,4,4′,5,5′-hexamethoxystilbene as described in Example 1.

Yield: 0.31 g (45%);

Flash point=270° C.

¹H-NMR (200 MHz, d₆-DMSO): δ 8.66 (s, 6H), 6.57 (s, 2H), 6.44 (s, 4H). ¹³C-NMR (50 MHz, d6-DMSO): δ 146.1, 132.9, 128.1, 125.8,105.2.

MS m/z 276 (M⁺, 100%).

Anal. (C₁₄H₁₂O₆) C, H.

Literature: Chemische Daten von Methoxystilbenen [Chemical Data of Methoxystilbenes]: J. Med. Chem. 45 (1), 1999, pp. 2671-2686

The pharmaceutical effectiveness of compounds according to the invention was examined:

### 1. Determination Series

Cell Culture

The human promyelocyte leukemia cell line HL-60 was purchased from the ATCC (American Type Culture Collection, Rockville, Md., USA). The cells were cultured in RPMI 1640 medium with 10% heat-inactivated fetal calf serum (FCS) (GIBCO, Grand Island Biological Co., Grand Island, N.Y., USA) and with 1% penicillin/streptomycin in a humidified atmosphere with 5% CO₂.

The cell numbers were determined by means of a microcell counter CC-108 (Sysmex, Kobe, Japan). For the tests, cells in the logarithmic growth phase were used.

Growth Inhibition Assay

Logarithmically-growing HL-60 cells were prepared in a density of 0.1×10⁶ cells/ml in tissue culture flasks and incubated with various concentrations of the stilbene derivatives to be examined. After 72 hours, the cells were counted by means of the microcell counter. The viability of the cells was determined by means of trypan blue staining. Numbers of viable cells were calculated as results.

COX (Human) Inhibitor Screening Assay

An immunoassay of the IBL Products Company, Hamburg, Germany was used for the determination of COX 1 and COX 2 activities. The assay quantitatively determines the prostaglandins F, E and D as well as thromboxan B of such prostaglandins, which are formed by the cyclooxygenase reaction. COX 1 and COX 2 were indicated as so-called IC₅₀, 50% enzyme inhibition, i.e., the substance concentration that inhibits 50% of the measured isoenzymes.

TABLE 1
Inhibiting action of stilbenes and resveratrol
on COX 1 and COX 2 activity:

| | IC₅₀(μM) | | COX2/COX1 |
|---|---|---|---|
| Substance | COX 1 | COX 2 | Ratio |
| 3,3′,4,5,5′-Pentamethoxystilbene | 10 | 0.6 | 0.06 |
| 3,3′,4,4′,5,5′-Hexamethoxystilbene | 10 | 0.5 | 0.05 |
| 3,4,4′,5-Tetrahydroxystilbene | 5 | 0.01 | 0.002 |
| 3,3′,5,5′-Tetrahydroxystilbene | 0.01 | <0.001 | <0.1 |
| 3,3′,4′,5-Tetrahydroxystilbene | 5 | 0.005 | 0.001 |
| 3,3′,4′,5,5′-Pentahydroxystilbene | 0.01 | 0.005 | 0.5 |
| 3,3′,4,4′,5,5′-Hexahydroxystilbene | 0.5 | 0.001 | 0.002 |
| 3,4′,5-Trihydroxystilbene | 0.5 | 0.5 | 1 |
| (Resveratrol) | | | |

As can be seen from Table 1, only the compounds according to the invention, but not resveratrol, itself COX 2, are selective (i.e., they show a significantly more effective action on COX 2 than on COX 1).

TABLE 2
Inhibiting action of compounds according to the invention
on the growth of HL-60 human promyelocyte leukemia cells:

| Substance | IC₅₀ (μM) |
|---|---|
| 3,3′,4,5,5′-Pentamethoxystilbene | 25 |
| 3,3′,4,4′,5,5′-Hexamethoxystilbene | >100 |
| 3,4,4′,5-Tetrahydroxystilbene | 9 |
| 3,3′,5,5′-Tetrahydroxystilbene | 12.5 |
| 3,3′,4′,5-Tetrahydroxystilbene | 9 |
| 3,3′,4′,5,5′-Pentahydroxystilbene | 10 |
| 3,3′,4,4′,5,5′-Hexahydroxystilbene | 4 |
| 3,4′,5-Trihydroxystilbene (Resveratrol) | 12 |

### Determination Series

Chemicals

3,4,5,3′,4′,5′-Hexahydroxystilbene was synthesized and used according to Example 11. Resveratrol is supplied by Sigma-Aldrich GmbH, Vienna, Austria. Both substances were diluted in DMSO.

Cell Culture

The human promyelocyte leukemia cell line HL-60 was purchased from the ATCC (American Type Culture Collection, Rockville, Md., USA). The cells were cultured in RPMI 1640 medium with 10% heat-inactivated fetal calf serum (FCS) (GIBCO, Grand Island Biological Co., Grand Island, N.Y., USA) and with 1% penicillin/streptomycin in a humidified atmosphere with 5% CO₂.

The cell numbers were determined by means of a microcell counter CC-108 (Sysmex, Kobe, Japan). For the tests, cells in the logarithmic growth phase were used.

Growth Inhibition Assay

Logarithmically-growing HL-60 cells were prepared in a density of 0.1×10⁶ cells/ml in tissue culture flasks and incubated with various concentrations of the resveratrol analogs. After 72 hours, the cells were counted by means of the microcell counter. The viability of the cells was determined by means of trypan blue staining. Numbers of viable cells were calculated as results.

### Analysis of the Intracellular dNTP (Deoxynucleoside Triphosphosphate) Pools By Means of High-Pressure Liquid Chromatography (HPLC)

The analysis of the intracellular dNTP concentrations was carried out according to the method of Garrett and Santi (Garrett, C.; Santi, D. V. A Rapid and Sensitive High-Pressure Liquid Chromatography Assay for Deoxyribonucleoside Triphosphates in Cell Extracts. Anal Biochem. 1979 Nov. 1; 99(2): 268-73.)

### Maximum Propidium Iodide Double-Coloring

HL-60 cells (0.1×10⁶/ml) were saturated in 25 cm² flasks and incubated for 24 hours with hexahydroxystilbene or resveratrol. Then, the cells were treated for one hour with Hoechst 33258 (HO, Sigma, St. Louis, Mo., USA) and propidium iodide (PI, Sigma, St. Louis, Mo., USA) (5 μg/ml and 2 μg/ml). Then, the cells were photographed by means of a fluorescence microscope, and the cells were divided morphologically into early apoptotic, late apoptotic and necrotic cells.

### Determination of the Cell Cycle Distribution

HL-60 cells were incubated in the presence of hexahydroxystilbene. After 24 hours, the cells were washed, centrifuged and fixed in alcohol. Then, iodide was colored with propidium and examined by means of FACS analysis. Then, the cell cycle phase distribution was calculated.

In untreated cells, 41.9% were in G0-G1, 43.8% in S and 14.3% in the G2-M phase of the cell cycle.

### Results of the 2^{nd} Determination Series

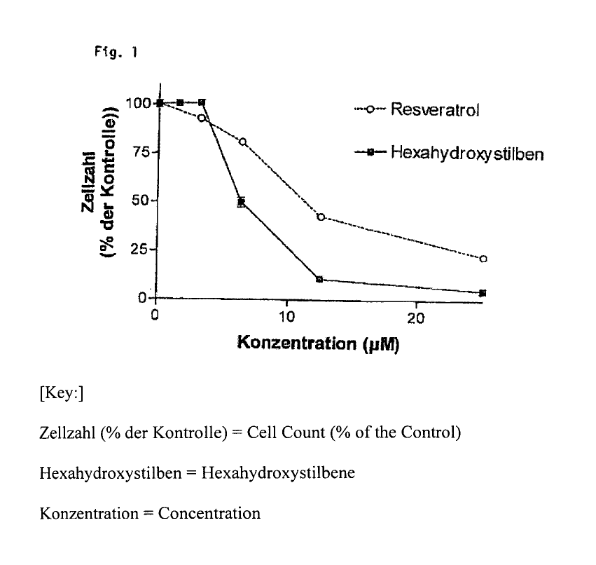

In the following description, reference is made to FIGS. 1 to 5. These show: Keys to the Figures

FIG. 1: Cytotoxic actions of resveratrol and hexahydroxystilbene in human HL-60 leukemia cells

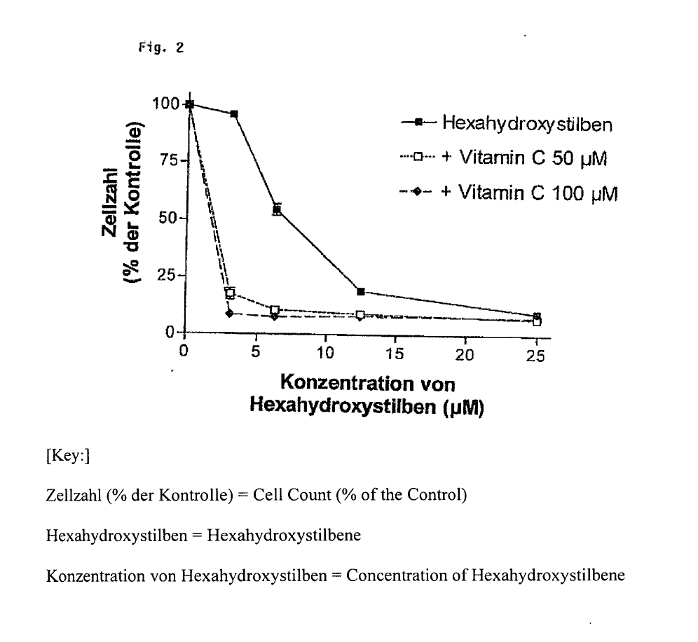

FIG. 2: Action of vitamin C and hexahydroxystilbene on the growth of HL-60 cells

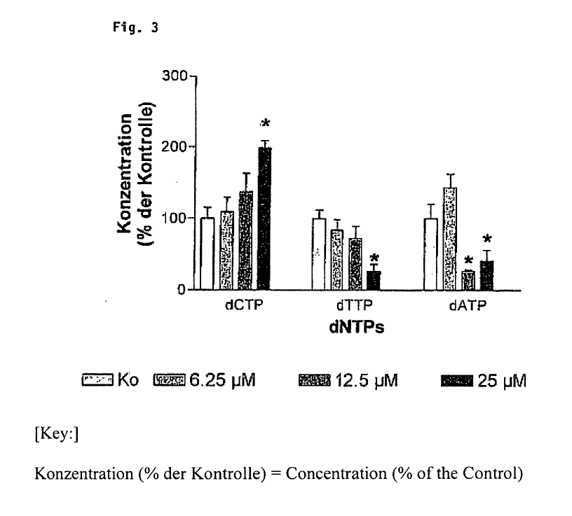

FIG. 3: Action of hexahydroxystilbene on intracellular dNTP levels of HL-60 cells.

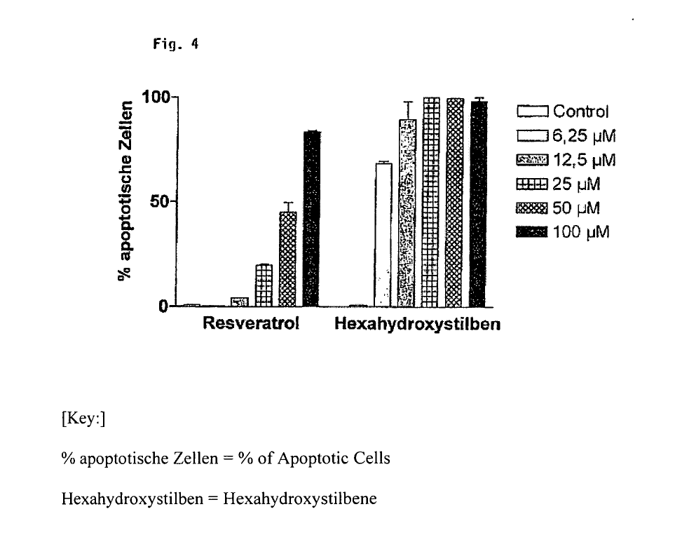

FIG. 4: Apoptosis induction by resveratrol and hexahydroxystilbene in HL-60 cells

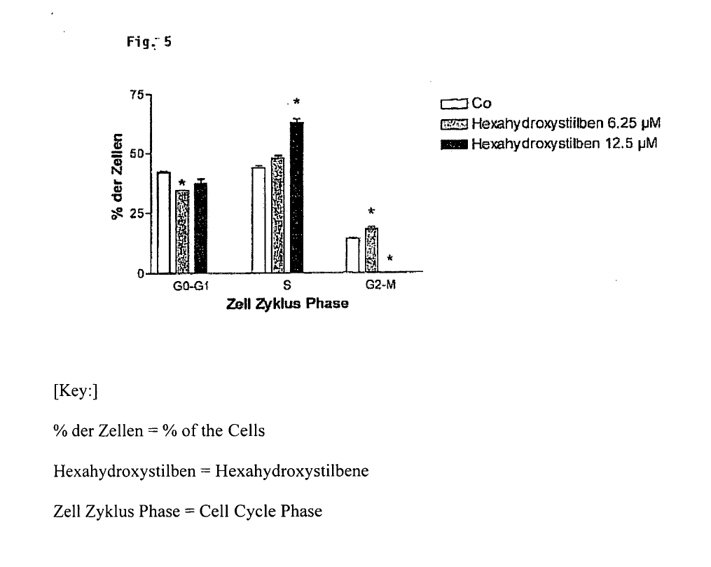

FIG. 5: Cell cycle phase distribution of HL-60 cells after treatment with hexahydroxystilbene

### Growth Inhibition Assay

The growth-inhibiting action of resveratrol or hexahydroxystilbene (“M8”) on HL-60 cells is plotted in FIG. 1. After 72 hours of incubation, resveratrol produced an IC50 (50% inhibition of cell growth) of 12 μM, while hexahydroxystilbene inhibited the cells in their growth with an IC50 value of 6.25 μM.

By adding 50 or 100 μM of vitamin C, the growth-inhibiting potential of the two substances, as plotted in FIG. 2, could be still further enhanced: for hexahydroxystilbene (“M8”), e.g., to an IC50 value of 2 μM. Vitamin C itself has shown no action on the cell growth in the concentrations used.

### Analysis of the Intracellular dNTP (Deoxynucleoside Triphosphosphate) Pools

The action of hexahydroxystilbene (“M8”) on the intracellular dNTP concentrations in HL-60 cells was examined. The study was performed as described above. The cells were incubated with 6.25, 12.5 and 25 μM of hexahydroxystilbene, and then the dNTP pools were determined. The dCTP pools increased to 110, 137 and 199% of the control values, while the dTTP pools dropped to 84, 72 and 27% of the starting values. The dATP concentrations dropped during treatment with 12.5 and 25 μM of hexahydroxystilbene to 27 and 41% of the starting values. The depletion of the dATP pools was especially impressive in the case of HT-29 human colon tumor cells; the dATP values dropped there as early as after treatment with 4 μM of the substance in an average of 1.5% of the starting values. There is scarcely any antiviral substance or substance that is used in the anti-tumor treatment that shows such impressive actions. The action of the substance can also be clarified by this impressive imbalance of the dNTP pools that are precursors of the DNA synthesis. The results of the experiment with HL-60 cells are plotted in FIG. 3.

### Induction of Apoptosis By Resveratrol Or Hexahydroxystilbene

It is known that resveratrol induces apoptosis in various tumor cells. We have compared the apoptotic action of resveratrol with that of hexahydroxystilbene (“M8”). HL-60 Leukemia cells were treated for 24 hours with various concentrations of resveratrol or hexahydroxystilbene (“M8”); then, the number of apoptotic cells was determined by means of maximum propidium iodide double-coloring. The results are plotted in FIG. 4. As can be seen from the figure, hexahydroxystilbene (“M8”) has induced apoptosis in these cells in the case of significantly lower concentrations than resveratrol. In the example, treatment with 6.25 μM of hexahydroxystilbene (“M8”) resulted in 68.5% of the cells for induction of apoptosis.

### Action of Hexahydroxystilbene On the Cell Cycle Distribution of HL-60 Leukemia Cells

As can be seen from FIG. 5, hexahydroxysilbene (“M8”) has significant influence on the cell cycle distribution of HL-60 cells. Treatment with hexahydroxystilbene has stopped the cells in S phase and thus produces an inhibition of the cell growth. Subsequently, after incubation with hexahydroxystilbene, a depletion of cells resulted in the G2-M phase of the cell cycle.

In Table 3 below, the levels of effectiveness of resveratrol and selected compounds according to the invention are presented:

Wachstumstrennung (72 h) von Stilben”-Derivaten in HL-60
und klonogenen Testreihen (7 d) in Prostata-Krebs-Zelllinien:

| Verbindung | Kode | HL-60 | | PC-3 | | LNCaP* | | DU-145 | |
|---|---|---|---|---|---|---|---|---|---|
| Resveratrol | | 12 | μM | 16 | μM | 5 | μM | 10 | μM |
| 3,5,3′,5′-tetramethoxy | M1 | uber 100 | μM | 35 | μM | 21 | μM | 25 | μM |
| 3,4,5,3′5′-pentamethoxy | M2 | 25 | μM | 21 | μM | uber 100 | μM | 68 | μM |
| 3,4,5,3′,4′,5′-hexamethoxy | M3 | uber 100 | μM | 5.5 | μM | 37 | μM | 12.5 | μM |
| 3,4,5,4′-tetramethoxy | M4 | 20 | μM | 3 | μM | 0.4 | μM | 0.4 | μM |
| 3,5,4′-trimethoxy | M5 | 5 | μM | 3 | μM | 6.25 | μM | 6 | μM |
| 3,4,3′,5′-tetramethoxy | M5A | 25 | μM | 35 | μM | 6.25 | μM | 30 | μM |
| 3,5,3′,5′-tetrahydroxy | M6 | 12.5 | μM | 16 | μM | 84 | μM | 17 | μM |
| 3,4,5,3′5′-pentahydroxy | M7 | 10 | μM | 5 | μM | 7.5 | μM | 23 | μM |
| 3,4,5,3′,4′,5′-hexahydroxy | M8 | 4 | μM | 2.5 | μM | 3.4 | μM | 25 | μM |
| 3,4,5,4′-tetrahydroxy | M9 | 9 | μM | 8 | μM | 9.5 | μM | 30 | μM |
| 3,4,3′,5′-tetrahydroxy | M10 | 9 | μM | 18 | μM | 33 | μM | 58 | μM |

* LNCaP-Zellen sind zu 5-alpha-dihydrotestosterone responsiv.
** jeweils “-Stilben”
[Key:]
Wachstumstrennung (72 h) von Stilben-Derivaten in HL-60 und klonogenen Testreihen (7 d) in Prostata-Kreb-Zelllinien: = Growth Separation (72 Hours) of Stilbene- Derivatives in HL-60 and Clonogenic Test Series (7 Days) in Prostate-Cancer
Cell Lines:
* LNCaP-Zellen sind zu 5-alpha-dihydrotestosterone responsive = LNCaP cells repond to 5-alpha-dihydrotestosterone
** jeweils “-Stilben” = ** respectively “-stilbene”

In summary, an embodiment of the invention can be depicted as follows:

Stilbene derivatives of general formula I 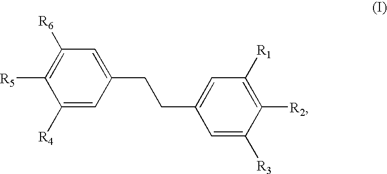
are described.

In the latter, at least four of the substituents R₁ to R₆ have a meaning different from hydrogen. The substituents are effective free-radical scavengers, anti-tumor active ingredients and selective cyclooxygenase-2 inhibitors.

1. Compound of general formula I 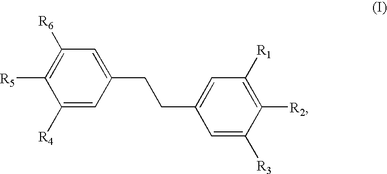
in which R₁ to R₆, which are the same or different, mean hydrogen, OH—, —OD or —OR₇, in which R₇ is a C₁ to C₃-alkyl group or a C₂ to C₄-carboxyl group, provided that at least four (4) of the substituents R₁ to R6 have a meaning different from hydrogen.

2. Compound according to claim 1, characterized in that R₇ is selected from the group that consists of methoxy, ethoxy and propoxy.

3. Compound according to claim 1, wherein R₇ is selected from the group that consists of formate and acetate.

4. Compound according to claim 1, wherein five (5) of the substituents R₁ to R₆ have a meaning different from hydrogen.

5. Compound according to claim 1, wherein six (6) of the substituents R₁ to R₆ have a meaning different from hydrogen.

6. Compound according to claim 1, in which R₁, R₂, R₃ and R₅ have a meaning different from hydrogen.

7. Compound according to claim 6, namely 3,3′,4,4′,5,5′-hexamethoxystilbene.

8. Compound according to claim 1, in which R₁, R₃, R₄ and R₆ have a meaning other than hydrogen.

9. Compound according to claim 8, namely 3,3′,5,5′-tetrahydroxystilbene.

10. Compound according to claim 1, in which R₁, R₃, R₄, and R₅ have a meaning different from hydrogen.

11. Compound according to claim 10, namely 3,3′,4′,5-tetrahydroxystilbene.

12. Compound according to claim 1, in which R₁, R₂, R₃, R₄ and R₅ have a meaning different from hydrogen.

13. Compound according to claim 12, namely 3,3′,4,4′,5-pentahydroxystilbene.

14. Compound according to claim 1, in which R₁, R₂, R₃, R₄ and R₆ have a meaning different from hydrogen.

15. Compound according to claim 14, namely 3,3′,4,5,5′-pentahydroxystilbene.

16. Compound according to claim 1, in which R₁, R₂, R₃, R₅ and R₆ have a meaning different from hydrogen.

17. Compound according to claim 16, namely 3,4,4′,5,5′-pentahydroxystilbene.

18. Compound according to claim 1, in which R₁, R₃, R₄, R₅ and R₆ have a meaning different from hydrogen.

19. Compound according to claim 18, namely 3,3′,4′,5,5′-pentahydroxystilbene.

20. Compound according to claim 1, wherein R₁ to R6 have a meaning different from hydrogen.

21. Compound according to claim 20, namely 3,3′,4,4′,5,5′-hexahydroxystilbene.

22. Compound according to claim 20, namely 3,3′,4,4′,5,5′-hexamethoxystilbene.

23. Pharmaceutical agents containing at least one of the compounds according to claim 1.

24. Pharmaceutical agents containing at least one of the compounds according to claim 1 as free-radical scavengers.

25. Pharmaceutical agents according to claim 24 containing 3,3′,4,4′,5,5′-hexahydroxystilbene and/or 3,3′,4,4′,5,5′-hexamethoxystilbene.

26. Pharmaceutical agents with anti-tumor action containing at least one compound of claim 1.

27. Pharmaceutical agents according to claim 26 containing 3,3′,4,4′,5,5′-hexahydroxystilbene and/or 3,3′,4,4′,5,5′-hexamethoxystilbene.

28. Pharmaceutical agents containing at least one of the compounds of claim 1 as selective cyclooxygenase-2 inhibitors.

29. Pharmaceutical agents according to claim 28 containing 3,3′,4,4′,5,5′-hexahydroxystilbene and/or 3,3′,4,4′,5,5′-hexamethoxystilbene.

30. Use of at least one compound of claim 1 for the production of pharmaceutical agents with free-radical scavengers, anti-tumor and cyclooxygenase-2 inhibiting action.
